(19) United States
(12) Patent Application Publication
(10) Pub. No.: US 2025/0032429 A1
(43) Pub. Date: Jan. 30, 2025

(54) 2-FLUORODESCHLOROKETAMINE FOR TREATMENT OF DEPRESSION, INCLUDING TREATMENT-RESISTANT DEPRESSION

(71) Applicant: CLEARMIND MEDICINE INC., Tel Aviv (IL)

(72) Inventor: Ezekiel Golan, Vancouver (CA)

(73) Assignee: CLEARMIND MEDICINE INC., Tel Aviv (IL)

(21) Appl. No.: 18/701,885

(22) PCT Filed: Oct. 18, 2022
(86) PCT No.: PCT/IB2022/060011

§ 371 Date: Apr. 16, 2024

Related U.S. Application Data
(60) Provisional application No. 63/256,728, filed on Oct. 18, 2021.

(51) Int. Cl. A61K 31/135; A61K 45/06; A61P 25/24

(52) U.S. Cl. CPC A61K 31/135; A61K 45/06; A61P 25/24

(57) ABSTRACT

Methods and compositions for the treatment of treatment-resistant depression are described. More specifically, the invention demonstrates that administering 2-fluorodeschloroketamine (2-FDCK) is effective to treat depression, especially to treat treatment-resistant depression.

### CROSS-REFERENCE TO RELATED APPLICATION

This application claims priority to and the benefit of U.S. Provisional Patent Application No. 63/256,728, filed Oct. 18, 2021 and titled “2-FLUORODESCHLOROKETAMINE FOR TREATMENT OF DEPRESSION, INCLUDING TREATMENT-RESISTANT DEPRESSION,” the entire content of which is incorporated herein by reference.

### FIELD OF THE INVENTION

The present invention relates to methods and compositions for the treatment of depression. More particularly, the invention relates to administration of 2-fluorodeschloroketamine (2-FDCK) to treat treatment-refractory or treatment-resistant depression.

### BACKGROUND

Depression, or Major Depressive Disorder (MDD), is among the most disabling of all medical disorders with a lifetime prevalence of approximately 17% (Kessler et al., Arch Gen Psychiatry. 62 (6): 593-602 (2005)). It frequently appears early in life, can run a chronic course, and adversely affect the prognosis of other medical illnesses, such as coronary vascular disease, diabetes, and osteoporosis.

Depression is characterized by depressed mood, and markedly diminished interest or pleasure in activities. Other symptoms include significant weight loss or weight gain, decrease or increase in appetite, insomnia or hypersomnia, psychomotor agitation or retardation, fatigue or loss of energy, feelings of worthlessness or excessive or inappropriate guilt, diminished ability to think or concentrate or indecisiveness, recurrent thoughts of death, suicidal ideation or suicidal attempts. A variety of somatic symptoms may also be present. Though depressive feelings are common, especially after experiencing setbacks in life, depressive disorder is diagnosed only when the symptoms reach a threshold and last at least two weeks. Depression can vary in severity from mild to very severe. It is most often episodic but can be recurrent or chronic. Some people have only a single episode, with a full return to premorbid function. However, more than 50 percent of those who initially suffer a single major depressive episode eventually develop another.

Depression is more common in women than in men. The point prevalence of unipolar depressive episodes is estimated to be 1.9% for men and 3.2% for women, and 5.8% of men and 9.5% of women will experience a depressive episode in a 12-month period. These prevalence figures vary across populations and may be higher in some populations. A World Health Organization study has reported that depression is the leading global cause of years of life lived with disability and the fourth leading cause of disability-adjusted life-years. Disability-adjusted life-years refers to the reduction in an individual's productive life, and is a measure that takes into account premature mortality (Murray et al., Lancet. 349 (9063): 1436-1442 (1997)).

The treatment of depression was revolutionized about a half-century ago by the serendipitous discovery of monoamine oxidase inhibitors and tricyclic antidepressants. Since then, the availability of a host of newer medications with better side effect profiles has greatly increased our ability to safely treat a significant percentage of patients. However, the newer medications are largely drugs that merely augment or otherwise potentiate the effects of the existing drugs by exerting their primary biochemical effects by increasing the intrasynaptic levels of monoamines.

Unfortunately, current medications for the treatment of depression take weeks to months to achieve their full effects and in the meantime, patients continue to suffer from their symptoms and continue to be at risk of self-harm as well as harm to their personal and professional lives. Indeed, the lag period of onset of action of several weeks of traditional antidepressants is recognized as a major limitation, resulting in considerable morbidity and high risk of suicidal behavior especially in the first 9 days of starting antidepressants (Jick et al., Jama. 292 (3): 338-343 (2004)). Pharmacological strategies that have rapid onset of antidepressant effects within hours or a few days and that are sustained would therefore have an enormous impact on public health. Patients with major depression respond to antidepressant treatment, but some of them do not improve after two consecutive courses of treatment with standard antidepressants and are considered to have what is termed as “Treatment-Resistant-Depression” (TRD).

TRD represents a heterogeneous state with likely multiple causal mechanisms. TRD patients exhibit the same diversity of symptoms, course, history and co-occurring conditions as for treatment-responsive MDD. However, very little is known about what distinguishes patients who do or do not respond to treatment. The extent to which individuals with TRD versus treatment-responsive MDD differ in etiology or pathophysiology remains mostly obscure, although there are several reports that a history of early life stress increases treatment-resistance (Bernet et al., Depress Anxiety. 9(4):169-74 (1999); Nanni et al., Am J Psychiatry. 169(2):141-51 (2012); Williams et al., Transl Psychiatry. 3; 6( ):e799 (2016)) and that individuals with TRD exhibit differences in brain circuit function (Dunlop et al., Am J Psychiatry. 174(6):533-545 (2017)). Nevertheless, the underlying mechanisms are not known.

Ketamine (a racemic mixture of the corresponding S- and R-enantiomers) is an N-methyl-D-aspartate (NMDA) receptor antagonist, with a wide range of effects in humans, including analgesia, anesthesia, hallucinations, dissociative effects, elevated blood pressure and bronchodilation. Ketamine is primarily used for the induction and maintenance of general anesthesia. Other uses include sedation in intensive care, analgesia (particularly in emergency medicine) and treatment of bronchospasms. Ketamine has also been shown to be efficacious in the treatment of depression (particularly in those who have not responded to other antidepressant treatment). In patients with major depressive disorders, ketamine has additionally been shown to produce a rapid antidepressant effect, acting within two hours. The U.S. Food and Drug Administration approved Spravato® (esketamine, a notation for S-Ketamine, the ‘S’ enantiomer of Ketamine) nasal spray, in conjunction with an oral antidepressant, for the treatment of depression in adults who have tried other antidepressant medicines, but have not benefited from them (i.e., TRD), in 2019. The S-ketamine enantiomer has higher potency or affinity for the NMDA reception and thus potentially allowing for lower dosages. However, because of the risk of serious adverse outcomes resulting from sedation and dissociation caused by Spravato® administration, and the potential for abuse and misuse of the drug, it is only available in the U.S. through a restricted distribution system, under a Risk Evaluation and Mitigation Strategy (REMS). Spravato® is self-administered by the patient under the supervision of a healthcare professional in a certified treatment center.

There remains a need to provide an effective treatment for depression, particularly in patients with treatment-refractory or treatment-resistant depression.

The inventor of the present invention surprisingly discovered that 2-(2-fluorophenyl)-2-(methylamino)cyclohexan-1-one (2-FDCK) unexpectedly has a flatter dose response curve compared to S-ketamine in treating patients with depression, specifically patients with TRD, leading to a potential reduction in side effects. Consequently, treating depression with 2-FDCK may allow for patient self-administration and possibility of eventually allowing the drug to be sold OTC at reduced cost to the patient compared to 2-FDCK as a prescription medication.

### SUMMARY OF THE DISCLOSURE

One aspect of the present invention provides a method for treating treatment-refractory or treatment-resistant depression, comprising administering to a patient in need thereof, a therapeutically effective amount of 2-(2-fluorophenyl)-2-(methylamino)cyclohexan-1-one (2-FDCK).

In another aspect, the present disclosure provides a method for the treatment of treatment-refractory or treatment-resistant depression comprising administering to a patient in need thereof, combination therapy with a therapeutically effective amount of 2-FDCK and at least one antidepressant, with a pharmaceutically acceptable carrier.

In some embodiments, the 2-FDCK is administered in an amount in the range from about 0.01 mg/kg to about 1.5 mg/kg. In other embodiments, the 2-FDCK is administered in an amount in the range from about 0.2 mg/kg to about 0.5 mg/kg. In yet other embodiments, the 2-FDCK is administered in an amount in the range from about 0.01 mg to about 1000 mg. In further embodiments, the 2-FDCK is administered in an amount in the range from about 1 mg to about 100 mg.

In some embodiments, the 2-FDCK forms a part of a composition, which further comprises at least one pharmaceutically acceptable carrier.

In some embodiments, the composition is administered intravenously. In other embodiments, the composition is administered intranasally. In other embodiments, the composition is administered orally.

In some embodiments, the 2-FDCK is administered in a unit dosage form composition. In some embodiments, the 2-FDCK is in the unit dosage form ranging from 1 mg to 1,000 mg.

In some embodiments, the method further comprises administering at least one antipressant. In certain embodiments, the at least one antipressant is selected from the group consisting of mono-amine oxidase inhibitors, tricyclics, serotonin reuptake inhibitors, serotonin noradrenergic reuptake inhibitors; noradrenergic and specific serotonergic agents and atypical antidepressants. In particular embodiments, the at least one antipressant is selected from the group consisting of imipramine, amitriptyline, desipramine, nortriptyline, doxepin, protriptyline, trimipramine, maprotiline, amoxapine, trazodone, bupropion, chlomipramine, fluoxetine, duloxetine, escitalopram, citalopram, sertraline, paroxetine, fluvoxamine, nefazadone, venlafaxine, milnacipran, reboxetine, lithium, mirtazapine, phenelzine, tranylcypromine, moclobemide, Kava-Kava, St. John's Wart, S-adenosylmethionine, thyrotropin releasing hormone, neurokinin receptor antagonists and triiodothyronine. In more particular embodiments, the at least one antipressant is selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, mirtazapine and bupropion.

In some embodiments, the 2-FDCK is self-administered or administered under the direction of a professional.

In some embodiments is provided a pharmaceutical composition for the treatment of treatment-refractory or treatment-resistant depression comprising 2-FDCK, optionally at least one antidepressant, and at least one pharmaceutically acceptable carrier.

### DETAILED DESCRIPTION

### Definitions

The following terms, unless otherwise indicated, shall be understood to have the following meanings:

As used herein, the terms “S-ketamine,” “S-ketamine hydrochloride,” and “esketamine” shall mean the (S)-enantiomer of ketamine, as its corresponding hydrochloride salt, a compound of formula

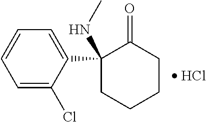 [SMILES: CN[C@]1(c2ccccc2Cl)CCCCC1=O ; "· HCl"]

also known as(S)-2-(2-chlorophenyl)-2-(methylamino)cyclohexanone hydrochloride.

As used herein, the term “2-FDCK,” “fluoroketamine,” and “2-fluorodeschloroketamine” is an analogue of ketamine where the chlorine group has been replaced by fluorine, a compound of formula

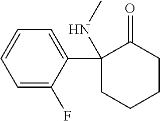 [SMILES: CNC1(c2ccccc2F)CCCCC1=O]

also known as 2-(2-fluorophenyl)-2-(methylamino)cyclohexan-1-one.

As used herein, the term “antidepressant” shall mean any pharmaceutical agent which can be used to treat depression. Suitable examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, citolapram, escitolapram, fluvoxamine, and the like; serotonin receptor antagonists such as nefazadone, and the like; serotonin noradrenergic reuptake inhibitors such as venlafaxine, milnacipran, desvenlafaxine, duloxetine and the like; noradrenergic and specific serotonergic agents such as mirtazapine, and the like; noradrenaline reuptake inhibitors such as reboxetine, edivoxetine and the like; atypical antidepressants such as bupropion, and the like; lithium, natural products such as Kava-Kava, St. John's Wort, and the like; dietary supplements such as s-adenosylmethionine, and the like; and neuropeptides such as thyrotropin-releasing hormone and the like; compounds targeting neuropeptide receptors such as neurokinin receptor antagonists and the like; and hormones such as triiodothyronine, and the like.

The term “chiral” refers to molecules, which have the property of non-superimposability of the mirror image partner.

The term “stereoisomers” as used herein, are compounds, which have identical chemical constitution, but differ with regard to the arrangement of the atoms or groups in space.

The term “enantiomers” as used herein refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

Where a compound exists in various tautomeric forms, the invention is not limited to any one of the specific tautomers, but rather includes all tautomeric forms.

The disclosure includes compounds of Formula I having all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

As used herein, including the claims, the singular forms of words, such as “a,” “an,” and “the,” include their corresponding plural references unless the context clearly dictates otherwise.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 mg” is intended to mean “about 10 mg”.

The term “about” when used before a numerical designation, e.g., temperature, time, amount, concentration, and such others, including a range, indicates approximations, which may vary, for example, by (+) or (−) 10%, 5%, 1%, or any subrange or subvalue there between, depending on the nature of the parameter and measurement. In some embodiments, the term “about” when used with regard to a dose amount means that the dose may vary by +/−10%. When a range of values is listed, it is intended to encompass each value and sub-range within the range.

The terms “comprises,” “comprising,” “includes,” “including,” “having” and their conjugates mean “including but not limited to.”

The term “consisting of” means “including and limited to.”

Unless otherwise indicated, the term “saline” means a 0.9 wt % aqueous sodium chloride solution.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “administration” or “administering” of the subject compound means providing a compound of the invention, a pharmaceutically acceptable salt, a pharmaceutically acceptable solvate, or solvate thereof to a subject in need of treatment.

The terms “composition” or “pharmaceutical composition,” as used herein, refers to a mixture of at least one compound, such as 2-FDCK, or a pharmaceutically acceptable salt thereof, with at least one and optionally more than one other pharmaceutically acceptable chemical components, such as carriers, stabilizers, diluents, dispersing agents, suspending agents, thickening agents, and/or excipients.

The terms “effective amount” or “therapeutically effective amount,” as used herein, refer to a sufficient amount of 2-FDCK being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

Wherein the present invention is directed to therapy with a combination of agents, “therapeutically effective amount” shall mean that amount of the combination of agents taken together so that the combined effect elicits the desired biological or medicinal response. For example, the therapeutically effective amount of combination therapy comprising 2-FDCK and a serotonin reuptake inhibitor would be the amount of 2-FDCK and the amount of the serotonin reuptake inhibitor that when taken together or sequentially have a combined effect that is therapeutically effective, and may have a combined effect that is synergistic. Further, it will be recognized by one skilled in the art that in the case of combination therapy with a therapeutically effect amount, the amount of each component of the combination individually may or may not be therapeutically effective.

Optimal dosages to be administered may be readily determined by those skilled in the art, and will vary with the particular compound or compounds used, the mode of administration, the strength of the preparation and the advancement of the disease condition. In addition, factors associated with the particular patient being treated, including patient's sex, age, weight, diet, time of administration and concomitant diseases/medications, will result in the need to adjust dosages.

In one embodiment, the 2-FDCK is administered at a dosage in the range from about 0.01 mg to about 1000 mg, or any amount or range therein, preferably from about 0.01 mg to about 500 mg, or any amount or range therein, preferably from about 0.1 mg to about 250 mg, or any amount or range therein. In another embodiment, the 2-FDCK is administered at a dosage in the range from about 0.01 mg to about 1000 mg, preferably selected from the group consisting of 0.01 mg, 0.025 mg, 0.05 mg, 0.1 mg, 0.5 mg, 1 mg, 5 mg, 10 mg, 25 mg, 50 mg, 100 mg, 150 mg, 200 mg, 250 mg, and 500 mg.

The term “pharmaceutically acceptable,” as used herein, refers to a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “carrier,” as used herein, refers to chemical compounds or agents that facilitate the incorporation of 2-FDCK into cells or tissues. The term “pharmaceutically acceptable carrier”, as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329; Remington: The Science and Practice of Pharmacy, 21^{st} Ed. Pharmaceutical Press 2011; and subsequent versions thereof). Except insofar as any conventional carrier is incompatible with the active ingredient, its use in the therapeutic or pharmaceutical compositions is contemplated.

The term “reduced” or “reduce” or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, at least 95%, at least 99% or up to and including a 100% decrease (i.e. substantially absent or below levels of detection), or any decrease between 10-100% as compared to a reference level, as that term is defined herein.

As used herein, the term “standard” or “reference” can simply be a reference that defines a baseline for comparison, such as a healthy individual(s) not suffering from depression.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As used herein, the term “subject” refers to a warm-blooded animal, such as a human that would benefit biologically, medically or in quality of life from the treatment. The subject can be mammals and non-mammals. Examples of the mammals include, but are not limited to, humans, chimpanzees, apes, monkeys, cattle, horses, sheep, goats, swine; rabbits, dogs, cats, rats, mice, guinea pigs, and the like. Examples of the non-mammals include, but are not limited to, birds, fish and the like. In one embodiment, the subject is human. It may be a human who has been diagnosed as in need of treatment for a disease or disorder disclosed herein.

As used herein, a subject is “in need of” a treatment if such subject would benefit biologically, medically or in quality of life from such treatment.

### Depression and Treatment Resistant Depression (TRD)

As used herein, the term “depression,” or “Major Depressive Disorder (MDD),” shall be defined to include major depressive disorder, unipolar depression, treatment-refractory depression, resistant depression, anxious depression, bipolar depression and dysthymia (also referred to as dysthymic disorder). Preferably, the depression is major depressive disorder, unipolar depression, treatment-refractory depression, resistant depression, anxious depression or bipolar depression.

Depression can be characterized by sadness, loss of interest in activities, and decreased energy. Other symptoms include loss of confidence and self-esteem, inappropriate guilt, thoughts of death and suicide, diminished concentration, and disturbance of sleep and appetite. A variety of somatic symptoms can also be present. Though depressive feelings are common, especially after experiencing setbacks in life, depressive disorder is diagnosed only when the symptoms reach a threshold and last at least two weeks. Depression can vary in severity from mild to very severe, and includes unipolar and bipolar depression, as well as seasonal affective disorder (SAD). Depression is typically also characterized into eight basic dimensions i.e., Pessimism, Weak Concentration, Sleep Problems, Anhedonia, Fatigue, Loneliness, Low Self-esteem, and Somatic Complaints to define the profile of children's and adolescents' depression. Depression can occur as an idiopathic disease (with no somatic disease associated with it), or it can be a psychiatric symptom of a somatic disorder, especially a number of neurodegenerative disorders.

Scales known in the art to be administered in assessing levels of depression include:
(1) Hamilton Depression Rating Scale 28-Item: primary outcome measure (Hamilton M. J., Neurol Neurosurg Psychiatry 1960; 23:56-62; Hamilton M., Br J Social Clin Psychology 1967; 6:278-296).
(2) Columbia-Suicide Severity Rating Scale (Posner K. et al., Am J Psychiatry. 2011; 168(12): 1266-77).
(3) Clinical Global Improvement Scale—Severity and Improvement (Guy W. Clinical Global Impression (CGI) ECDEU Assessment manual for Psychopharmacology. Rockville, Md.: U.S. Dept Health Education and Welfare 1976).
(4) Quick Inventory of Depressive Symptoms, Self-Report version (Trivedi M. H. et al., Psychol Med. 2004; 34(1):73-82).
(5) Concise Health Risk Tracking (Trivedi M. H. et al., J Clin Psychiatry. 2011; 72(6):757-64).
(6) MGH Cognitive and Physical Functioning Questionnaire (Fava M. et al., Psychother Psychosom. 2009; 78(2):91-7).
(7) Quality of Life Enjoyment and Satisfaction Questionnaire (Endicott J. et al., Psychopharmacol Bull. 1993; 29(2):321-6).
(8) Brief Psychiatric Rating Scale (Andersen J. et al. Psychopathology. 1989; 22(2-3):168-76; Hafkenscheid A., Acta Psychiatr Scand. 1991; 84 (3): 294-300).
(9) Only at baseline and end of Phase 3-Neurocognitive Test Battery: immediate and delayed verbal recall, speed of comprehension, digit span forward and backward, N-back test, and trail-making task (Trandafir A. et al., Schizophr Res. 2006; 81 (2-3): 217-26).

As used hrerein, the term “treatment-refractory or treatment-resistant depression” and the abbreviation “TRD” shall be defined as major depressive disorder that does not respond to adequate courses of at least two antidepressants, preferably two or more antidepressants, more preferably two to three, antidepressants.

One skilled in the art will recognize that the failure to respond to an adequate course of a given antidepressant may be determined retrospectively or prospectively. In an embodiment, at least one of the failures to respond to an adequate course of antidepressant is determined prospectively. In another embodiment, at least two of the failures to respond to an adequate course of antidepressant are determined prospectively. In another embodiment, at least one of the failures to respond to an adequate course of antidepressant is determined retrospectively. In another embodiment, at least two of the failures to respond to an adequate course of antidepressant are determined retrospectively.

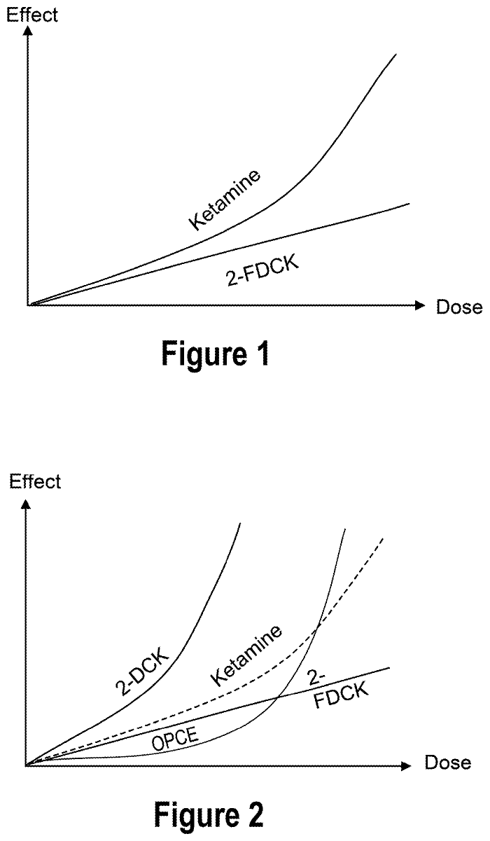

While not intending to be bound to any specific theory, the inventor has observed that the dose-response curve for 2-FDCK is flatter than the dose-response curve for Ketamine when administered in the same way (see FIG. 1). The difference in dose response is substantial enough to be used in the treatment of depression in a way that eliminates many of the shortcomings, for example, adverse side effects, that are inherent in current practices using Ketamine for treating the same condition. Due to the reduced risk of experiencing debilitating side effects several advantages flow from the use of 2-FDCK for TRD, including, shorter turnaround times for clinic visits, the possibility of eventually allowing patient self-administration (further reducing costs, and/or the possibility of approval of 2-FDCK to be sold over the counter. In contrast, as shown in FIG. 2, other arylcyclohexamines, such as 2-DCK and OPCE, have dose-response curves that start rising sharply at low doses making them less suitable as alternative therapeutics for Ketamine.

### Pharmaceutical Compositions and Combination

Pharmaceutical compositions of the present disclosure comprise at least 2-FDCK together with one or more pharmaceutically acceptable carriers.

One skilled in the art will recognize that, both in vivo and in vitro trials using suitable, known and generally accepted cell and/or animal models are predictive of the ability of a test compound to treat or prevent a given disorder.

Wherein the present invention is directed to the administration of a combination, the compounds may be co-administered simultaneously, sequentially, separately or in a single pharmaceutical composition. Where the compounds are administered separately, the number of dosages of each compound given per day, may not necessarily be the same, e.g. where one compound may have a greater duration of activity, and will therefore, be administered less frequently. Further, the compounds may be administered via the same or different routes of administration, and at the same or different times during the course of the therapy, concurrently in divided or single combination forms. The instant invention is therefore understood as embracing all regimens of simultaneous or alternating treatment and the term “administering” is to be interpreted accordingly.

As used herein, the terms “co-therapy”, “combination therapy”, “adjunctive treatment”, “adjunctive therapy” and “combined treatment” shall mean treatment of a patient in need thereof by administering 2-FDCK in combination with one or more antidepressant(s), and further, optionally in combination with one or more atypical antipsychotics wherein the esketamine and the antidepressant(s) are administered by any suitable means, simultaneously, sequentially, separately or in a single pharmaceutical formulation. Where the 2-FDCK and the antidepressant(s) are administered in separate dosage forms, the number of dosages administered per day for each compound may be the same or different. The 2-FDCK and the antidepressant(s) may be administered via the same or different routes of administration. Examples of suitable methods of administration include, but are not limited to, oral, intravenous (iv), intranasal (in) intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The 2-FDCK and the antidepressant(s) may be administered according to simultaneous or alternating regimens, at the same or different times during the course of the therapy, concurrently in divided or single forms.

Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, 2-FDCK, and optionally, at least one antidepressant, as the active ingredient(s) are intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful, and the like, of from about 0.01 mg to about 1000 mg or any amount or range therein, and may be given at a dosage of from about 0.01 mg/kg to about 1.5 mg/kg, or any amount or range therein, preferably from about 0.01 mg/kg/day to about 0.75 mg/kg, or any amount or range therein, preferably from about 0.05 mg/kg to about 0.5 mg/kg, or any amount or range therein, preferably from about 0.1 mg/kg to about 0.5 mg/kg, or any amount or range therein, of each active ingredient. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 mg to about 1,000 mg, or any amount or range therein, of each active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating treatment-refractory or treatment-resistant depression described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein; preferably from about 0.05 mg to about 500 mg of the compound, or any amount or range therein, of each active ingredient, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, esketamine, optionally in combination with at least one antidepressant, as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In one aspect of the invention, provided herein is a method of treating treatment-refractory or treatment-resistant depression comprising administering to a subject in need thereof a therapeutically effective amount of 2-fluorodeschloroketamine (2-FDCK).

In some embodiments, the 2-FDCK is administered in an amount of about 0.01 mg/kg to about 1.5 mg/kg. In some embodiments, the 2-FDCK is administered in an amount of about 0.1 mg/kg to about 0.5 mg/kg. In some embodiments, the 2-FDCK is administered in an amount of about 0.1 mg/kg. In some embodiments, the 2-FDCK is administered in an amount of about 0.15 mg/kg. In some embodiments, the 2-FDCK is administered in an amount of about 0.2 mg/kg. In some embodiments, the 2-FDCK is administered in an amount of about 0.25 mg/kg. In some embodiments, the 2-FDCK is administered in an amount of about 0.3 mg/kg. In some embodiments, the 2-FDCK is administered in an amount of about 0.35 mg/kg. In some embodiments, the 2-FDCK is administered in an amount of about 0.4 mg/kg. In some embodiments, the 2-FDCK is administered in an amount of about 0.45 mg/kg. In some embodiments, the 2-FDCK is administered in an amount of about 0.5 mg/kg.

In some embodiments, the 2-FDCK is administered in the range from about 0.01 mg to about 1000 mg. In some embodiments, the 2-FDCK is administered in the range from about 5 mg to about 100 mg. In some embodiments, the 2-FDCK is administered in an amount of about 5 mg. In some embodiments, the 2-FDCK is administered in an amount of about 10 mg. In some embodiments, the 2-FDCK is administered in an amount of about 15 mg. In some embodiments, the 2-FDCK is administered in an amount of about 20 mg. In some embodiments, the 2-FDCK is administered in an amount of about 25 mg. In some embodiments, the 2-FDCK is administered in an amount of about 30 mg. In some embodiments, the 2-FDCK is administered in an amount of about 35 mg. In some embodiments, the 2-FDCK is administered in an amount of about 40 mg. In some embodiments, the 2-FDCK is administered in an amount of about 45 mg. In some embodiments, the 2-FDCK is administered in an amount of about 50 mg. In some embodiments, the 2-FDCK is administered in an amount of about 55 mg. In some embodiments, the 2-FDCK is administered in an amount of about 60 mg. In some embodiments, the 2-FDCK is administered in an amount of about 65 mg. In some embodiments, the 2-FDCK is administered in an amount of about 70 mg. In some embodiments, the 2-FDCK is administered in an amount of about 75 mg. In some embodiments, the 2-FDCK is administered in an amount of about 80 mg. In some embodiments, the 2-FDCK is administered in an amount of about 85 mg. In some embodiments, the 2-FDCK is administered in an amount of about 90 mg. In some embodiments, the 2-FDCK is administered in an amount of about 95 mg. In some embodiments, the 2-FDCK is administered in an amount of about 100 mg.

In some embodiments, the 2-FDCK forms a part of a composition which further comprises a pharmaceutically acceptable carrier. In one embodiment, the composition is administered intravenously. In one embodiment, the composition is administered intranasally. In one embodiment, the composition is administered orally.

In some embodiments, the 2-FDCK is administered in a unit dosage form composition. In a further embodiment, the 2-FDCK is in the unit dosage form ranging from XX mg to YY mg. In some embodiments, the 2-FDCK is in a unit dosage form of XX mg. In some embodiments, the 2-FDCK is in a unit dosage form of QQ mg. In some embodiments, the 2-FDCK is in a unit dosage form of WW mg. In some embodiments, the 2-FDCK is in a unit dosage form of YY mg.

In another aspect of the disclosure, the method further comprises administering at least one antipressant. In some embodiments, the at least one antipressant includes, but not limited to, mono-amine oxidase inhibitors, tricyclics, serotonin reuptake inhibitors, serotonin noradrenergic reuptake inhibitors; noradrenergic and specific serotonergic agents and atypical antidepressants. In some embodiments, the at least one antipressant includes, but not limited to, imipramine, amitriptyline, desipramine, nortriptyline, doxepin, protriptyline, trimipramine, maprotiline, amoxapine, trazodone, bupropion, chlomipramine, fluoxetine, duloxetine, escitalopram, citalopram, sertraline, paroxetine, fluvoxamine, nefazadone, venlafaxine, milnacipran, reboxetine, lithium, mirtazapine, phenelzine, tranylcypromine, moclobemide, Kava-Kava, St. John's Wart, S-adenosylmethionine, thyrotropin releasing hormone, neurokinin receptor antagonists and triiodothyronine. In some embodiments, the at least one antipressant includes, but not limited to, phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, mirtazapine and bupropion.

In another aspect of the present disclosure, provided herein is a pharmaceutical composition for the treatment of treatment-refractory or treatment-resistant depression comprising 2-FDCK, optionally at least one antidepressant, and at least one pharmaceutically acceptable carrier.

The examples and preparations provided below further illustrate and exemplify the compounds as disclosed herein and methods of preparing such compounds. It is to be understood that the scope of the present disclosure is not limited in any way by the scope of the following examples and preparations.

Various embodiments and aspects of the present invention as delineated hereinabove and as claimed in the claims section below find experimental support in the following examples.

### EXAMPLES

The examples and preparations provided below further illustrate and exemplify the compounds as disclosed herein and methods of preparing such compounds. It is to be understood that the scope of the present disclosure is not limited in any way by the scope of the following examples and preparations.

### Example 1:2-FDCK Show Antidepressant Efficacy in the Mouse Forced Swim Test

### Summary

Male 10 week C57BL/6N mice (Taconic) are acclimated to a holding facility one week prior to testing. A two-day FST paradigm is employed, in which mice are exposed to a 10 minute test in absence of drug, and are tested the following day in presence of drug in a 6 minute test. 2-FDCK is administered intraperitoneally (i.p.) in saline vehicle 30 minutes prior to FST. All animals are drug naïve and not previously exposed to behavioral testing.

### Materials and Methods

### Animals

Male C57BL6N/Tac mice 10 weeks of age are housed four mice per cage under a 12:12 h light/dark cycle and are allowed to acclimate for approximately one week prior to behavioral procedures. Food and water is provided ad libitum. All procedures followed the National Institutes of Health Guide for the Care and Use of Laboratory Animals.

### Drug Treatment

2-FDCK is dissolved in a saline vehicle (0.9% sodium chloride). For the 2-FDCK dose response experiment, mice receive vehicle or varying doses of 2-FDCK i.p. 30 minutes prior to behavior testing.

### Behavioural Procedures

Mice are tested in a two day forced swim test (FST) procedure. On day 1, all mice receive mock intraperitoneal vehicle (i.p.) injections to acclimate to the injection procedure and, 30 minutes later, are placed into one of five identical cylindrical chambers (24 cm×15 cm) filled approximately halfway with warm water (26)±2° for 10 minutes with no data collected. On day 2, mice receive i.p. injections 30 minutes prior to being placed in a cylinder for a 6 minute session and immobility time is scored during the final 4 minutes. Mice receiving 2-FDCK exhibit shorter immobility times relative to mice receiving vehicle alone.

### Example 2: Dose-Response Curves for Selected Arylcyclohexamines

For safety reasons, when exploring novel psychoactives—the logarithmic scale is adopted for initial dosing to get an idea of whether or not a compound is active and if so, at what dosage ranges.

Following this initial experimentation with OPCE, 2-DCK, and 2-FDCK (Ketamine was later added to the study for reference) it was clear that dosages of 3 mg, 15 mg, and 30 mg, correspondingly, were effective as lower thresholds for further exploration. It was decided to multiply the lower threshold doses by 2, 3 & 4 to explore the effects of higher doses on the mind. Every several days a portion of one of the compounds was measured out on an Ohaus Scout balance scale and prepared for administration via nasal spray using a refillable nasal spray bottle.

The goals were to score a “+” ranking for effect and keep an open mind to look out for any other noteworthy observations. The Shulgin “+” based rating system (SHULGIN, A. T., & SHULGIN, A. (1991). Pihkal: a chemical love story. Berkeley, CA, Transform Press, page 964) for psychedelic experiences was chosen for a yearlong exploration of these various arylcylohexamines. Although the target family of molecules and the type of experience were different from the rating systems' original design, the scale's simplicity yields itself particularly well to this kind of exploration for a few reasons:
(1) The experience rendered by novel psychedelics is often ineffable. The language for discussing new states of consciousness must be developed and agreed upon—and this process, quite naturally happens after a large number of people have experienced the compound and develop a discourse about its effects.
(2) The arylcyclohexamines are often motor inhibiting or motor altering making writing difficult or impossible during the experience.
(3) The dissociative nature of the effect of many arylcyclohexamines does not allow for lengthy time windows of focus to relate an intricate or detailed decision.

It was therefore desirable to use the simple +, ++, +++, ++++ as 1, 2, 3, 4 to rate intensity of experience without seeking an objective measure for intensity.

The data collected from this study is summarized in the following Table:

TABLE 1

| | 2-DCK | 2-DCK | OPCE | OPCE | 2-FDCK | 2-FDCK | Ketamine | Ketamine |
|---|---|---|---|---|---|---|---|---|
| | dosage | effect | dosage | effect | dosage | effect | dosage | effect |
| October 5 | | | | | 31 | 1 | | |
| October 10 | | | | | 58 | 2 | | |
| October 14 | | | | | 29 | 1 | | |
| October 18 | | | | | 57 | 2 | | |
| October 22 | | | | | 62 | 2 | | |
| October 23 | | | | | 90 | 3 | | |
| October 24 | | | | | 91 | 3 | | |
| October 27 | | | | | 33 | 1 | | |
| October 28 | | | 6 | 1 | | | | |
| November 3 | | | 3 | 1 | | | | |
| November 7 | | | 6 | 1 | | | | |
| November 8 | | | | | 88 | 3 | | |
| November 9 | | | | | 114 | 4 | | |
| November 13 | | | | | 116 | 4 | | |
| November 14 | | | | | 124 | 4 | | |
| November 15 | | | 3 | 1 | | | | |
| November 23 | | | 3 | 1 | | | | |
| November 27 | | | | | 122 | 4 | | |
| December 3 | | | | | 35 | 1 | | |
| December 9 | | | | | 55 | 2 | | |
| December 13 | | | 6 | 1 | | | | |
| January 9 | | | 9 | 2 | | | | |
| January 14 | | | 9 | 2 | | | | |
| January 18 | | | 3 | 1 | | | | |
| January 23 | | | 12 | 4 | | | | |
| February 1 | | | 4 | 1 | | | | |
| February 5 | | | 6 | 1 | | | | |
| February 10 | 29 | 3 | | | | | | |
| February 14 | 31 | 3 | | | | | | |
| February 17 | 45 | 4 | | | | | | |
| February 21 | 46 | 4 | | | | | | |
| February 24 | 16 | 1 | | | | | | |
| February 27 | 16 | 1 | | | | | | |
| March 2 | 29 | 3 | | | | | | |
| March 5 | 15 | 1 | | | | | | |
| March 6 | | | 9 | 2 | | | | |
| March 9 | | | 11 | 4 | | | | |
| March 10 | | | 12 | 4 | | | | |
| March 13 | | | 12 | 4 | | | | |
| March 16 | 17 | 1 | | | | | | |
| March 19 | 61 | 4 | | | | | | |
| March 22 | 18 | 1 | | | | | | |
| March 23 | 28 | 3 | | | | | | |
| March 27 | 44 | 4 | | | | | | |
| March 31 | 57 | 4 | | | | | | |
| April 4 | 58 | 4 | | | | | | |
| April 7 | 62 | 4 | | | | | | |
| April 10 | | | | | | | 32 | 1 |
| April 12 | | | | | | | 31 | 1 |
| April 16 | | | | | | | 59 | 2 |
| April 19 | | | | | | | 28 | 1 |
| April 22 | | | | | | | 58 | 2 |
| April 28 | | | | | | | 62 | 2 |
| April 29 | | | | | | | 90 | 4 |
| May 3 | | | | | | | 92 | 4 |
| May 6 | | | | | | | 31 | 1 |
| May 9 | | | | | | | 120 | 4 |
| May 12 | | | | | | | 31 | 1 |
| May 15 | | | | | | | 55 | 2 |
| May 18 | | | | | | | 89 | 3 |
| May 21 | | | | | | | 114 | 4 |
| May 22 | | | | | | | 114 | 4 |
| May 25 | | | | | | | 122 | 4 |
| May 28 | 33 | 3 | | | | | | |
| May 29 | 43 | 4 | | | | | | |
| May 30 | 42 | 4 | | | | | | |
| May 31 | | | 3 | 1 | | | | |
| June 3 | | | 11 | 4 | | | | |
| June 6 | | | 4 | 1 | | | | |
| June 7 | | | 9 | 2 | | | | |
| June 9 | | | | | 28 | 1 | | |
| June 11 | | | | | 31 | 1 | | |
| June 13 | | | | | 61 | 2 | | |
| June 15 | | | | | 30 | 1 | | |
| June 17 | | | | | 57 | 2 | | |
| June 19 | | | | | 61 | 2 | | |
| June 21 | | | | | 91 | 3 | | |
| June 23 | | | | | 90 | 3 | | |
| June 25 | | | | | 29 | 1 | | |
| June 27 | | | | | 32 | 1 | | |
| June 29 | | | | | 31 | 1 | | |
| July 1 | | | | | 58 | 2 | | |
| July 3 | | | | | 29 | 1 | | |
| July 5 | | | | | 57 | 2 | | |
| July 9 | | | | | 62 | 2 | | |
| July 10 | | | | | 90 | 3 | | |
| July 11 | | | | | 91 | 3 | | |
| July 12 | | | | | 33 | 1 | | |
| July 13 | | | 6 | 1 | | | | |
| July 14 | | | 3 | 1 | | | | |
| July 15 | | | 6 | 1 | | | | |
| July 16 | | | | | 88 | 3 | | |
| July 17 | | | | | 114 | 4 | | |
| July 18 | | | | | 116 | 4 | | |
| July 19 | | | | | 124 | 4 | | |
| July 20 | | | 3 | 1 | | | | |
| July 23 | | | | | 35 | 1 | | |
| July 26 | | | 9 | 2 | | | | |
| July 28 | | | 3 | 1 | | | | |
| July 30 | | | 4 | 1 | | | | |
| August 1 | 29 | 3 | | | | | | |
| August 4 | 46 | 4 | | | | | | |
| August 6 | 16 | 1 | | | | | | |
| August 7 | 29 | 3 | | | | | | |
| August 12 | | | 12 | 4 | | | | |
| August 13 | 17 | 1 | | | | | | |
| August 14 | 61 | 4 | | | | | | |
| August 15 | | | | | | | 62 | 2 |
| August 16 | | | | | | | 90 | 4 |
| August 17 | | | | | | | 92 | 4 |
| August 19 | 58 | 4 | | | | | | |
| August 20 | 62 | 4 | | | | | | |
| August 21 | | | | | | | 32 | 1 |
| August 22 | | | | | | | 31 | 1 |
| August 26 | 18 | 1 | | | | | | |
| August 27 | 28 | 3 | 1 | | | | | |
| August 28 | 44 | 4 | | | | | | |
| August 29 | | | | | | | 31 | 1 |
| September 2 | | | | | | | 89 | 3 |
| September 3 | | | | | | | 114 | 4 |
| September 4 | | | | | | | 114 | 4 |
| September 5 | | | | | | | 122 | 4 |
| September 8 | 42 | 4 | | | | | | |
| September 9 | | | 3 | 1 | | | | |
| September 18 | | | | | 61 | 2 | | |
| September 23 | | | | | 31 | 1 | | |
| September 24 | | | | | 58 | 2 | | |
| September 25 | | | | | 29 | 1 | | |
| September 29 | | | | | 91 | 3 | | |
| September 30 | | | | 33 | 1 | | | |
| October 1 | | 6 | 1 | | | | | |
| October 3 | | 6 | 1 | | | | | |
| October 4 | | | | | 88 | 3 | | |
| October 6 | | | | | 116 | 4 | | |
| October 7 | | | | | 124 | 4 | | |
| October 9 | | | | | 35 | 1 | | |
| October 10 | | | 9 | 2 | | | | |
| October 14 | 46 | 4 | | | | | | |
| October 15 | 16 | 1 | | | | | | |
| October 16 | 29 | 3 | | | | | | |
| October 22 | | | | | | | 92 | 4 |
| October 23 | 58 | 4 | | | | | | |
| October 24 | 62 | 4 | | | | | | |
| October 25 | | | | | | | 32 | 1 |
| October 26 | | | | | | | 31 | 1 |

When the dosages are adjusted to units that make all dose/response curves fit on the same graph then running a spline through the data points gives us curves that resemble, qualitatively, the curves appearing in FIG. 2.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. One skilled in the art will readily appreciate that the specific methods and results discussed in the Example are merely illustrative of the invention as described more fully in the claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

We claim:

1. A method of treating depression, optionally treatment-refractory or treatment-resistant depression, comprising administering to a subject in need thereof a therapeutically effective amount of 2-fluorodeschloroketamine (2-FDCK).

2. The method of claim 1, wherein the 2-FDCK is administered in an amount in the range from about 0.01 mg/kg to about 1.5 mg/kg.

3. The method of claim 2, wherein the 2-FDCK is administered in an amount in the range from about 0.2 mg/kg to about 0.5 mg/kg.

4. The method of claim 1, wherein the 2-FDCK is administered in an amount in the range from about 0.01 mg to about 1000 mg.

5. The method of claim 1, wherein the 2-FDCK is administered in an amount in the range from about 1 mg to about 100 mg.

6. The method of claim 1, wherein the 2-FDCK forms a part of a composition, which further comprises at least one pharmaceutically acceptable carrier.

7. The method of claim 6, wherein the composition is administered intravenously.

8. The method of claim 6, wherein the composition is administered intranasally.

9. The method of claim 6, wherein the composition is administered orally.

10. The method of claim 1, wherein the 2-FDCK is administered in a unit dosage form composition.

11. The method of claim 10, wherein the 2-FDCK is in the unit dosage form ranging from 1 mg to 1,000 mg.

12. The method of any one of claims 1-11, further comprising administering at least one antipressant.

13. The method of claim 12, wherein the at least one antipressant is selected from the group consisting of mono-amine oxidase inhibitors, tricyclics, serotonin reuptake inhibitors, serotonin noradrenergic reuptake inhibitors; noradrenergic and specific serotonergic agents and atypical antidepressants.

14. The method of claim 12, wherein the at least one antipressant is selected from the group consisting of imipramine, amitriptyline, desipramine, nortriptyline, doxepin, protriptyline, trimipramine, maprotiline, amoxapine, trazodone, bupropion, chlomipramine, fluoxetine, duloxetine, escitalopram, citalopram, sertraline, paroxetine, fluvoxamine, nefazadone, venlafaxine, milnacipran, reboxetine, lithium, mirtazapine, phenelzine, tranylcypromine, moclobemide, Kava-Kava, St. John's Wart, S-adenosylmethionine, thyrotropin releasing hormone, neurokinin receptor antagonists and triiodothyronine.

15. The method of claim 12, wherein the at least one antipressant is selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, mirtazapine and bupropion.

16. The method of any of claims 1-15, wherein the 2-FDCK is self-administered or administered under the direction of a professional.

17. A pharmaceutical composition for use in the method of any of claims 1-16 comprising 2-FDCK, optionally at least one antidepressant, and at least one pharmaceutically acceptable carrier.
